SEQUENCE LISTING (4 sequences)

SEQ ID NO: 1 - DNA, 1473 bp, Unknown
  Bacillus amyloliquefaciens
gacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatgcttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttggggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttttggagccagccgccgaaggtgggacagatgattggggtg

SEQ ID NO: 2 - DNA, 1472 bp, Unknown
  bacillus siamensis
gacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtg

SEQ ID NO: 3 - DNA, 1472 bp, Unknown
  Bacillus velezensis
gacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtg

SEQ ID NO: 4 - DNA, 1472 bp, Unknown
  Bacillus tequilensis
gacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtg

(19) United States
(12) Patent Application Publication
(10) Pub. No.: US 2022/0372432 A1
(43) Pub. Date: Nov. 24, 2022

(54) BIOLOGICAL MICROBIAL TREATING AGENT FOR RADIOACTIVE MATERIAL REMOVAL

(71) Applicant: Seungchang YANG, Muan-gun (KR)

(72) Inventor: Seungchang YANG, Muan-gun (KR)

(21) Appl. No.: 17/417,266

(22) PCT Filed: Dec. 24, 2018
(86) PCT No.: PCT/KR2018/016608

§ 371 Date: Jun. 22, 2021

(30) Foreign Application Priority Data
Dec. 23, 2018 (KR) 10-2018-0168026

(51) Int. Cl. C12N 1/20; B09C 1/10; C02F 3/34; G21F 9/18; G21F 9/30

(52) U.S. Cl. CPC C12N 1/205; B09C 1/10; C02F 3/341; G21F 9/18; G21F 9/30; C12R 2001/07

(57) ABSTRACT

The present invention provides a biological microbial treating agent, for radioactive material removal, comprising at least one type of microorganism selected from Bacillus amyloliquefaciens KS-R01, Bacillus siamensis KS-R02, Bacillus velezensis KS-R03 and Bacillus tequilensis KS-R04.

### TECHNICAL FIELD

The present invention relates to a biological microbial treating agent and, more particularly, to a biological microbial treating agent for radioactive material removal, which has resistance to radioactivity, has an excellent ability to remove radioactive materials, and thus restores polluted water quality or soil, thereby preventing environmental contamination and restoring environment.

### BACKGROUND ART

In addition, if the atomic nucleus of an unstable element decays by itself, radioactivity is released from the inside. This decay capacity per unit time is called radioactivity. In addition, the atomic nucleus with such property is called a radionuclide, and a substance containing the radionuclide is called a radioactive material.

In the natural world, the atomic nuclei of about 40 elements with relatively large atomic numbers, including uranium and radium, belong to this radionuclide, and those artificially made radioactive by atomic nucleus reactions include about 1,000 radionuclides, including an element number 1 hydrogen and an element number 104 rutherfordium.

Meanwhile, the radioactivity is usefully applied to the medical field to kill cancer cells and prevent cancer cells from proliferating around the cancer cells. However, excessive exposure to the human body causes severe damage to tissues and organs in a short time. Depending on an exposure dose, acute syndromes such as cerebrovascular syndrome, gastrointestinal (GI) syndrome, and hematopoietic syndrome may appear, and the onset and progress of symptoms due to radioactive exposure depends on the amount of radioactivity.

Thus, in case of materials that contain or may contain radioactive materials, it is essential to determine whether to dispose of those materials or not after checking the presence and concentration of radioactive materials and measuring the nuclides. In particular, when dismantling nuclear power plants, a large amount of soil or concrete waste are generated, and these wastes contain radioactive materials at various concentrations, such as soil having radioactivity at an extremely low level near a deregulation level and soil having radioactivity at a high level, including uncontaminated soil, but a soil treatment method has so far relied on simple disposal.

Furthermore, radon (Rn-222) is produced upon the radioactive decay of uranium which is contained in a trace amount (7.4 to 74 Bq/kg) in soil, rocks, and minerals in the crust, and construction materials made thereof.

Radioactivity present in nature was first discovered Becquerel in 1896. Out of about 340 species of isotopes present nature, now 70 species are known to emit radioactively, and heavy elements, especially those with an atomic number greater than 82 (Pb), are all radioactive isotopes. Radon belongs to a series of radioactive isotopes generated while continuing nuclear transformation with uranium as a starting point, which are called uranium series, and the most important isotope in the uranium series is radium (Ra-226) , which exists in nature along with uranium and reportedly occurs a lot in granite areas.

In Korea, radon is detected in most areas that belong to the granite zone. In certain areas, a large amount of radon exceeding several times a standard value is detected, which becomes a social problem, but there is little method for dealing with this problem.

### DISCLOSURE

### Technical Problem

The present invention has been proposed to solve the problems of the related art as above, and an object of the present invention is to provide a biological microbial treating agent for radioactive material removal, which has resistance to radioactivity, has an excellent ability to remove radioactive materials, and. thus treats polluted water quality or soil, and further radioactive wastes generated from the operation of nuclear power plants and radioactive wastes generated from military and special industries, thereby preventing environmental contamination and restoring environment.

### TECHNICAL SOLUTION

The technical object of the present invention as described above is achieved by the following solutions.
(1) A biological microbial treating agent for radioactive material removal, including at least one type of microorganism selected from Bacillus amyloliquefaciens KS-R01, Bacillus siamensis KS-R02, Bacillus velezensis KS-R03 and Bacillus tequilensis KS-R04.
(2 ) The biological microbial treating agent of claim 1, including Bacillus amyloliquefaciens KS-R01, Bacillus siamensis KS-R02, Bacillus velezensis KS-R03 and Bacillus tequilensis KS-R04.
(3) A method for removing radioactive materials by treating water or soil contaminated with radioactive materials with the microbial treating agent of claim 1 or 2.

### ADVANTAGEOUS EFFECTS

The present invention has resistance to radioactivity, has an excellent ability to remove radioactive materials, and thus restores polluted water quality or soil, thereby preventing environmental contamination and restoring environment.

### DESCRIPTION OF DRAWINGS

FIG. 1 is a view showing the results of showing a degree of colony formation of four selected strains according to the present invention by radioactive irradiation.

FIG. 2 is a survival curve of four selected strains according to the present invention by radioactive irradiation.

### MODE FOR INVENTION

Hereinafter, the content, of the present invention will be described in more detail as follows.

As described. above, the present invention may provide a biological microbial treating agent for radioactive material removal, which. contains at least type of microorganism selected from Bacillus amylcliquefaciens KS-R01, Bacillus siamensis KS-R02, Bacillus velezensis KS-R03, and Bacillus tequilensis KS-R04.

For the microorganisms used above in the present invention, the present invention may provide a biological microbial treating agent for radioactive material removal, which includes at least one type of microorganism selected from Bacillus amylcliquefaciens KS-R01 (KCTC 13558BP) , Bacillus siamensis KS-R02 (KCTC 13559BP), Bacillus velezensis KS-R03 (KCTC 13560BP), and Bacillus tequilensis KS-R04 (KCTC 13561BP).

The biological microbial treating agent according to the present invention is not particularly limited, but may be mixed with each microbial strain at a ratio of 1 to 30 wt %. The biological microbial treating agent according to the present invention may have the four types of microorganisms mixed to exert a synergistic effect and environmentally remove radioactive materials as the agent is directly put into places contaminated with radioactive materials, such as ocean, river, lake or soil, other articles contaminated with radiation, or the like.

Bacillus amyloliquefaciens KS-R01 (KCTC 13558BP), Bacillus siamensis KS-R02 (KCTC 13559BP), Bacillus velezensis KS-R03 (KCTC 13560BP) and Bacillus tequilensis KS-R04 (KCTC 13561BP), which form the biological microbial treating agent according to the present invention, were deposited to the Gene Bank in the Korea Research Institute of Bioscience and Biotechnology on Jun. 21, 2018.

The biological microbial treating agent according to the present invention, containing the above-described microorganisms, may be prepared by the following method.

The 0.001 to 0.02 wt % of microbial starters consisting of the above microbial strains (at least one of the four species), 2 to 5 wt % of rice bran, 2 to 4 wt % of molasses, and 2 to 4 wt % of brown sugar may be mixed with the rest of water to make it 100 wt %, and cultured for 15 to 21 days by repeating a process of performing an aeration at 100 to 500 L/h for 1 to 3 hours at intervals of 2 to 6 hours 2 to 6 times a day while maintaining a temperature of 20 to 25° C., thereby preparing a liquid microbial agent. The above conditions such as the aeration, etc., may be those optimized as a result of the inventor's research, and those skilled in the art will appreciate that some modifications may be made to the above conditions, but such modifications do not depart from the scope of the present invention.

After preparing the liquid microbial agent as above, a mixed raw material including 20 to 30 wt % of one or more raw materials for cultivation selected from the group consisting of dried powder of rice bran, agricultural by-products, and agricultural and forest products, and 70 to 80 wt % of the liquid microbial agent may be prepared, and the inoculation step of inoculating 0.01 to 0.1 wt % of the microbial starters according to the present invention may be performed.

The biological microbial treating agent of the present invention may use rice bran, agricultural by-products, agricultural and forest products, or the like, which are relatively easy to obtain and have a low cost burden, as a raw material for cultivation of microorganisms. For the raw material for cultivation, the rice bran, agricultural by-products or agricultural and forest products may be used alone or in combination, but may be preferably formed at a composition ratio of 35 to 45 wt % of rice bran, 25 to 35 wt % of agricultural by-products, and 20 to 30 wt % of agricultural and forest products. It is preferable that the raw material for cultivation is pulverized with 100 mesh in advance and used in a powder form. The mixed and pulverized raw materials for cultivation may be put into a direct warmed incubator, after which moisture may be adjusted with a liquid fermenting agent to maintain an environment suitable for microbial fermentation, and then inoculated with the mixed microbial starters.

After performing the inoculation step, the inoculated mixed raw materials may be subjected to a high-temperature cultivation step of culturing at 80 to 85° C. for 2 to 8 hours. As described above, the method for preparing a biological microbial treating agent of the present invention may have a feature of being cultured at an ultra-high temperature of 80° C. or higher. Considering that normal microbial cultivation is performed in the range of 20 to 40° C., it can be understood that the biological microbial treating agent of the present invention is performed under ultra-high temperature conditions. In particular, in one embodiment of the present invention, the raw materials for cultivation, inoculated with the mixed starters, may be cultured at an ultra--high temperature of 80 to 85° C. for 2 to 8 hours while stirring at a speed of 30 to 80 rpm/min. In this case, the cultivation may be performed at a temperature of 80° C. or higher so as to suppress the proliferation of unnecessary microorganisms, but may be preferably performed at 85° C. or lower to maintain the activity of the polymicrobes according to the present invention.

The method of inputting the biological microbial treating agent that may be prepared as described above is to directly spray from a culture tank containing the biological microbial treating agent according to the present invention onto a radioactive contaminated area by using a sprayer, etc., or to directly input to the contaminated area simply by installing a fixed injection tank. The biological microbial treating agent to be inputted may vary depending on a radioactive contaminant, but in general may be made into a 10 to 30-fold dilution of the microbial preparation stock solution to make an activating solution, and then inputted in an appropriate amount several times at an interval of 1 to 3 days.

The stock solution of the microbial preparation may be prepared by adding 0.1 to 1.0 parts by weight of the microbial preparation, preferably 0.2 to 0.7 parts by weight, and most preferably 0.5 parts by weight, based on 100 parts by weight of the water, mixing with 0.5 to 2.0 parts by weight of molasses, preferably 0.8 to 1.2 parts by weight, and most preferably 1 part by weight, and culturing with an aeration for about 72 hours while maintaining at 18 to 25° C. The activating solution may be formed, for example, by adding water 10 times the amount of the microbial preparation stock solution at a weight ratio so as to make a composition containing 0.2 wt % of molasses when preparing a 10-fold activating solution. After that, the activating solution may be obtained by culturing with an aeration. for about 48 hours while maintaining at 18 to 25° C.

Hereinafter, the content of the present invention will be described through the following examples and experimental examples. However, these examples are provided only for the purpose of illustrating the present invention in more detail, and thus the scope of the present invention is not limited thereto.

### Experimental Example 1 Isolation Of Radiation-Resistant Strains

1. Radioactive resistance of KS50 complex microbial agent

KS50 starters, powder products, liquid products and liquid products, which are complex microbial agents developed by Keonnong Co., Ltd., were subjected to irradiation with different Co-60 gamma rays of 0, 4, 6, 8, 10 kGy, so as to isolate surviving microorganisms.

As shown in Tables 1 and 2, for KS50 starters (F2), powder products (F3), and liquid products (F4), which were not irradiated with radioactivity, a total of 3.3×10⁷, 5.8×10⁶, and 4.2×10⁶ CFU were present TSA medium, respectively, and 1.0×10⁶, 4.8×10⁶, and 6.3×10⁶ CFU were present in YM medium.

The maximum irradiation dose viable in the TSA medium was 10 kGy for F2, 4 kGy for F3, and 10 kGy for F4, in which 5.0×10¹, 1.9×10³ and 5 colonies were detected, respectively. In the YM medium, the maximum irradiation dose was confirmed to be 4 kGy for F2 with 5.0×10¹ CFU, 6 kGy for F3 with 5.0×10² CFU, and 6 kGy for F4 with 1.0×10² CFU.

From these results, it was confirmed that the KS50 complex microbial starters and products contain a number of microorganisms that are resistant to radioactivity.

There was a difference in the number of viable cells depending on the irradiation dose according to the composition of the medium, but the number of viable cells decreased as the irradiation dose increased. In particular, when the irradiation dose was increased to 4 kGy, the number of viable cells was rapidly decreased. Accordingly, the irradiation dose for screening efficient radiation-resistant microorganisms was selected to be 4 kGy or more.

TABLE 1
Measurement of viable cell counts in TSA medium (CFU/mL)

| | Irradiation dose (kGy) | | | | | | | |
|---|---|---|---|---|---|---|---|---|
| TSA(BD) | 0 | 0.5 | 1 | 2 | 4 | 6 | 8 | 10 |
| F2 | 3.3 × 10⁷ | 1.0 × 10⁶ | 2.5 × 10⁶ | 6.3 × 10⁶ | 1.2 × 10⁴ | 1.0 × 10² | 10 × 10² | 5.0 × 10¹ |
| F3 | 5.8 × 10⁶ | 1.5 × 10⁶ | 3.8 × 10⁵ | 6.3 × 10⁴ | 1.9 × 10³ | ND | ND | ND |
| F4 | 4.2 × 10⁶ | 6.8 × 10⁵ | 2.9 × 10⁵ | 4.5 × 10⁴ | 3.5 × 10² | 5.0 × 10¹ | 5.0 | 5.0 |

* ND: not detected

TABLE 2
Measurement of viable cell counts in YM medium (CFU/mL)

| | Irradiation dose (kGy) | | | | | | | |
|---|---|---|---|---|---|---|---|---|
| YM(BD) | 0 | 0.5 | 1 | 2 | 4 | 6 | 8 | 10 |
| F2 | 1.0 × 10⁶ | 2.5 × 10⁶ | 1.5 × 10⁶ | 1.5 × 10⁵ | 5.0 × 10¹ | ND | ND | ND |
| F3 | 4.8 × 10⁶ | 8.0 × 10⁶ | 8.5 × 10⁶ | 2.5 × 10⁶ | 4.5 × 10³ | 5.0 × 10² | ND | ND |
| F4 | 6.3 × 10⁶ | 1.2 × 10⁶ | 6.0 × 10⁵ | 2.8 × 10⁴ | 1.5 × 10² | 1.0 × 10² | ND | ND |

*ND: not detected

2. Isolation of radiation--resistant strains from TSA complex microbial agent

The surviving strains were isolated from the first Co-60 (Cobalt 60) radioactive irradiation, and then re-irradiated at an irradiation dose of 4-15 kGy so as to isolate and identify the surviving strains. A total of 22 types of microorganisms were isolated on the TSA medium and a total of 14 types of microorganisms were isolated on the YM medium, and their survival was confirmed by irradiation with gamma rays at an irradiation dose of 4, 6, 8, 10, 11, 12, 13, and 15 kGy. As shown in Tables 3 and 4, it appears that the survival was achieved even at an irradiation of 8 kGy for T11-6k, 11 kGy for T12-11k, 4 kGy for T14-4k, 4 kGy for T15-4k, 11 kGy for T16-11k, 6 kGy for T17-6k, 6 kGy for T18-6k, and 8 kGy for T19-8k, while the survival was achieved even at an irradiation of 6 kGy for Y6-6k, 8 kGy for Y7-8k, and 6 kGy for Y11-6k. As described above, 9 types of microorganisms that survived at an irradiation dose of 6 kGy were isolated, 5 types of microorganisms that survived at 8 kGy were isolated, and 2 types of microorganisms that survived at 11 kGy were isolated.

The microorganisms that survived at an irradiation dose of 4 kGy or higher were found to be microorganisms which were detected in liquid products cultured in liquid medium using the starters of KS50complex microbial agent.

TABLE 3
Radioactive resistance of strains isolated from TSA medium (CFU/mL)

| | First radioactive | | | | | | | | | | |
|---|---|---|---|---|---|---|---|---|---|---|---|
| | irradiation | | Second radioactive | | | | | | | | |
| | | Viable cell | irradiation (kGy) | | | | | | | | |
| Name | Information | count | 4 | 6 | 8 | 10 | 11 | 12 | 13 | 15 | Id |
| T1 | F2-10 kGy | 5.0 × 10¹ | — | — | — | — | — | — | — | — | |
| T2 | F2-6 kGy | 5.0 × 10² | — | — | — | — | — | — | — | — | |
| T3 | F2-6 kGy | 5.0 × 10² | — | — | — | — | — | — | — | — | |
| T4 | F2-6 kGy | 5.0 × 10² | — | — | — | — | — | — | — | — | |
| T5 | F2-4 kGy | 4.5 × 10³ | — | — | — | — | — | — | — | — | |
| T6 | F2-4 kGy | 4.5 × 10³ | — | — | — | — | — | — | — | — | |
| T7 | F2-4 kGy | 4.5 × 10³ | — | — | — | — | — | — | — | — | |
| T8 | F2-4 kGy | 2.0 × 10⁴ | — | — | — | — | — | — | — | — | |
| T9 | F2-4 kGy | 2.0 × 10⁴ | — | — | — | — | — | — | — | — | |
| T10 | F2-4 kGy | 2.0 × 10⁴ | — | — | — | — | — | — | — | — | |
| T11 | F4-8 kGy | 2.5 × 10¹ | ◯ | ◯ | ◯ | — | — | — | — | — | T11-6k |
| T12 | F4-8 kGy | 2.5 × 10¹ | ◯ | ◯ | ◯ | ◯ | ◯ | — | — | — | T12-11k |
| T13 | F4-8 kGy | 2.5 × 10¹ | ◯ | — | — | — | — | — | — | — | |
| T14 | F4-8 kGy | 2.5 × 10¹ | ◯ | — | — | — | — | — | — | — | T14-4k |
| T15 | F4-6 kGy | 5.0 × 10¹ | ◯ | — | — | — | — | — | — | — | T15-4k |
| T16 | F4-6 kGy | 5.0 × 10¹ | ◯ | ◯ | ◯ | ◯ | ◯ | — | — | — | T16-11k |
| T17 | F4-6 kGy | 2.0 × 10² | ◯ | ◯ | — | — | — | — | — | — | T17-6k |
| T18 | F4-6 kGy | 2.0 × 10² | ◯ | ◯ | — | — | — | — | — | — | T18-6k |
| T19 | F4-6 kGy | 2.0 × 10² | ◯ | ◯ | ◯ | — | — | — | — | — | T19-8k |
| T20 | F3-8 kGy | 2 | — | — | — | — | — | — | — | — | |
| T21 | F3-8 kGy | 2 | — | — | — | — | — | — | — | — | |
| T22 | F2-8 kGy | 2.5 × 10² | — | — | — | — | — | — | — | — | |

TABLE 4
Radioactive resistance of microorganisms isolated from YM medium (CFU/mL)

| | First radioactive | | | | | | | | | | |
|---|---|---|---|---|---|---|---|---|---|---|---|
| | irradiation | | Second radioactive | | | | | | | | |
| | | Viable cell | irradiation | | | | | | | | |
| Name | Information | count | 4 | 6 | 8 | 10 | 11 | 12 | 13 | 15 | Id |
| Y1 | F4-8 kGy | <9 | — | — | — | — | — | — | — | — | |
| Y2 | F4-8 kGy | <9 | — | — | — | — | — | — | — | — | |
| Y3 | F4-8 kGy | <9 | — | — | — | — | — | — | — | — | |
| Y4 | F4-8 kGy | <9 | — | — | — | — | — | — | — | — | |
| Y5 | F4-6 kGy | 1 × 10² | — | — | — | — | — | — | — | — | |
| Y6 | F4-6 kGy | 1 × 10² | ◯ | ◯ | | — | — | — | — | — | Y6-6k |
| Y7 | F4-4 kGy | 1.5 × 10² | ◯ | ◯ | ◯ | ◯ | ◯ | — | — | — | Y7-8k |
| Y8 | F4-4 kGy | 1.5 × 10² | — | — | — | — | — | — | — | — | — |
| Y9 | F4-4 kGy | 1.5 × 10² | — | — | — | — | — | — | — | — | — |
| Y10 | F4-4 kGy | 1.5 × 10² | — | — | — | — | — | — | — | — | — |
| Y11 | F4-4 kGy | 1.5 × 10² | ◯ | ◯ | — | — | — | — | — | — | Y11-6k |
| Y12 | F4-4 kGy | 1.5 × 10² | — | — | — | — | — | — | — | — | — |
| Y13 | F3-2 kGy | 2.5 × 10⁶ | — | — | — | — | — | — | — | — | — |
| Y14 | F2-2 kGy | 1.5 × 10⁵ | — | — | — | — | — | — | — | — | — |

* Information marked: Sample type-irradiation dose* TSA medium: Tryptic soy agar, cultured at 35° C. for 24 hours
* YM medium: Yeast and mold media, cultured at 30° C. for 48 hours
* Colony production marked - “◯”
* A final ID mark for strain identification was written based on the second radioactive irradiation.

3. Identification of radiation-resistant microorganisms

A total of 36 types of strains were isolated from samples that survived the first radioactive irradiation (Tables 3 and 4), and the radioactive irradiation was performed at an irradiation dose ranging from 4 to 15 kGy, so as to identify microorganisms based on 16S rRNA gene sequence for 28 types of surviving microorganisms.

As a result of analyzing a total of 28 types of microorganisms (Table 5), it was composed of 4 types of microorganisms. Finally, as shown in. Table 6, it was identified that the microorganisms surviving at an irradiation dose of 11 kGy are Bacillus amyloliquefaciens (T16-11k) and Bacillus siamensis (T12-11k), the microorganism surviving at 8 kGy is Bacillus velezensis (T19-8k), and the microorganism surviving at 6 kGy is Bacillus tequilensis (Y11-6k). Radiation-resistant microorganisms appeared all as Bacillus bacteria, and Bacillus is known to have resistance to extreme environments by forming resistant spores in extreme environments such as ultraviolet rays, high temperatures, low temperatures, high pressures, etc. Microorganisms that survived at an irradiation dose of 4 kGy or more are considered to have resistance by resistant spores. The analyzed 16S nucleotide sequence is as shown in Tables 7 and 10.

TABLE 5
Results of 16S identification for a total of 28 strains

| | | | | Diff/ |
|---|---|---|---|---|
| No. | ID | Stain name | Similarity | Total |
| 1 | T16-11k | Bacillus amyloliquefaciens | 99.86 | 2/1472 |
| 2 | T12-11k | Bacillus siamensis | 99.86 | 2/1472 |
| 3 | T19-8k | Bacillus velezensis | 99.86 | 2/1403 |
| 4-1 | T16-8k-b-1 | Bacillus velezensis | 99.86 | 2/1403 |
| 4-2 | T16-8k-b-2 | Bacillus amyloliquefaciens | 99.8 | 3/1472 |
| 4-3 | T16-8k-b-3 | Bacillus amyloliquefaciens | 99.86 | 2/1471 |
| 4-4 | T16-8k-b-4-a | Bacillus amyloliquefaciens | 99.86 | 2/1471 |
| 4-5 | T16-8k-b-4-b | Bacillus amyloliquefaciens | 99.86 | 2/1471 |
| 5-1 | T12-8k-b-1 | Bacillus velezensis | 99.86 | 2/1403 |
| 5-2 | T12-8k-b-2 | Bacillus velezensis | 99.86 | 2/1403 |
| 5-3 | T12-8k-b-3 | Bacillus velezensis | 99.86 | 2/1403 |
| 6 | T19-6k | Bacillus velezensis | 99.86 | 2/1403 |
| 7 | T18-6k | Bacillus velezensis | 99.86 | 2/1403 |
| 8 | T17-6k | Bacillus siamensis | 99.93 | 1/1472 |
| 9 | T11-6k | Bacillus amyloliquefaciens | 99.8 | 3/1472 |
| 10-1 | T15-4k-a-1 | Bacillus tequilensis | 99.93 | 1/1472 |
| 10-2 | T15-4k-a-2 | Bacillus tequilensis | 99.93 | 1/1472 |
| 10-3 | T15-4k-b | Bacillus tequilensis | 99.93 | 1/1472 |
| 10-4 | T15-4k-c-1 | Bacillus tequilensis | 99.93 | 1/1472 |
| 10-5 | T15-4k-c-2 | Bacillus tequilensis | 99.93 | 1/1472 |
| 10-6 | T15-4k-d | Bacillus tequilensis | 99.93 | 1/1472 |
| 10-7 | T15-4k-e | Bacillus tequilensis | 99.93 | 1/1472 |
| 10-8 | T15-4k-f | Bacillus tequilensis | 99.93 | 1/1472 |
| 11 | T14-4k | Bacillus amyloliquefaciens | 99.8 | 3/1472 |
| 12 | Y7-8k | Bacillus amyloliquefaciens | 99.8 | 3/1472 |
| 13 | Y11-6k | Bacillus tequilensis | 99.93 | 1/1472 |
| 14 | Y6-6k | Bacillus amyloliquefaciens | 99.8 | 3/1472 |
| 15 | Y7-4k | Bacillus amyloliquefaciens | 99.8 | 3/1472 |

TABLE 6
Four types of non-overlapping strains with high
radioactive resistance among 28 strains

| No. | ID | Strain name | Similarity | Diff/Total |
|---|---|---|---|---|
| 1 | T16-11k | Bacillus amyloliquefaciens | 99.86 | 2/1472 |
| 2 | T12-11k | Bacillus siamensis | 99.86 | 2/1472 |
| 3 | T19-8k | Bacillus velezensis | 99.86 | 2/1403 |
| 4 | Y11-6k | Bacillus tequilensis | 99.93 | 1/1472 |

4. Radioactive resistance of selected strains

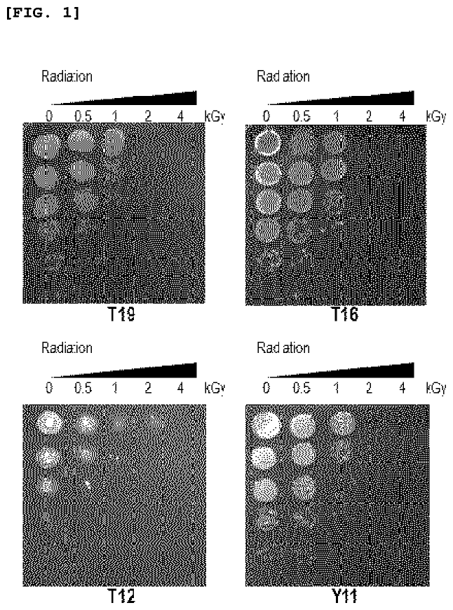

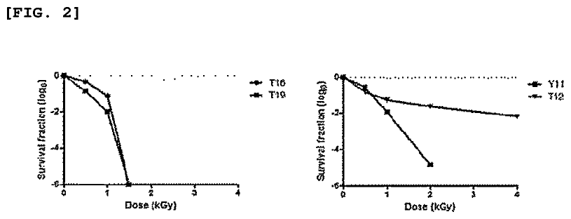

As a result of smearing four types of strains irradiated with radioactivity at an irradiation dose of 0.5 to 4 kGy and examining a growth pattern (FIG. 1), it was observed that T16 and T19 strains do not for a colony at an irradiation dose of or more, whereas Y11 and T12 strains have a colony surviving even at a dose of 2 kGy. Y11 strain showed a survival rate about 0.001% at 2 kGy, and T12 strain showed a survival rate of 2% at 2 kGy and about 0.7% at 4 kGy. In addition, as a result of confirming a survival trend according to the irradiation dose (FIG. 2), it was shown that T16 strain is −1.1, Y11 strain is −1.9, and T19 strain is −2.5, but T12 strain is −0.48, showing a definitely higher survival curve depending on doses than other strains.

In order to confirm the quantitative radioactive resistance, it was attempted to calculate the D10 value of the strain (Table 7). Based on the survival rate, a trend line value of a growth curve for each strain could be calculated as follows.

TABLE 7
Radioactive resistance of isolated microorganisms

| Sample | b | a | D10 value (kGy) |
|---|---|---|---|
| T16 | 0.078 | −1.13 | 0.95 |
| T19 | 0.053 | −2.00 | 0.53 |
| Y11 | 0.135 | −1.94 | 0.58 |
| T12 | −0.46 | −0.48 | 1.13 |

As a result of calculating a D10 value (an irradiation dose at which 90% of strains die) based on the survival curve of each. strain, the T12 strain. showed the D10 value=1.13, which was a higher value than that of other strains. In conclusion, it means that the T12 strain has higher radioactive resistance than other strains. The radioactive susceptibility of microorganisms may vary depending on the microorganism's various species, growth states, environments, or the like. Gram-positive bacteria and resistant spores of the genus Clostridium or Basillus, and some radiation-resistant microorganisms may exhibit high radioactive resistance with a D10 value of 1 kGy or more. In contrast, food poisoning bacteria that do not form spores (Salmonella, Staphylococcus, Listeria, Escherichia, etc.) may have low radioactive resistance and thus show a D10 value of around 0.5 kGy. It has been reported that there is a sterilization effect of about 10⁻⁶ level by radioactive irradiation of 3 kGy, which is an irradiation dose of radioactive sterilization for general food.

### Example 1

The 0.01 kg of Bacillus amyloliquefaciens KS-R01 (KCTC 13558BP) starter, 4 kg of rice bran, 2 kg of molasses, and 4 kg of brown sugar were mixed with water suitable for drinking water to make it a weight of 100 kg, and then cultured for 21 days by repeating a process of performing an aeration at 100 L/h for 2 hours at intervals of 4 hours 4 times a day while maintaining a temperature of 20 to 25° C., thereby preparing a liquid microbial agent. The liquid fermenting agent had a total number of bacteria of 4.3=10⁷ cfu/g.

Apart from the above, 65 kg of rice bran and 35 kg of agricultural by-products which were pulverized in advance were added to an incubator with a mixing function and mixed for 30 minutes. The 30 kg of the liquid fermenting agent was added to the mixed raw materials to adjust a moisture concentration to 70%, and then the microbial mixed starters according to the present invention were inoculated at 0.01% of the total weight the raw materials for cultivation. The inoculated raw materials for cultivation were cultured for four hours in an incubator at a ultra-high internal temperature of 80 to 85° C. while stirring at 30 to 80 rpm/min, so as to prepare a complex microbial preparation of the present invention.

The 1 ton of water was added to 5 kg of the complex microbial preparation, and 10 kg of molasses was added thereto and cultured at 25° C. for 72 hours along with aeration so as to obtain a stock solution. Water was added 20 times the amount of the stock. solution at a weight ratio, and 0.2 wt % of molasses was added to obtain a 20-fold dilution of the stock solution for an activating solution.

### Example 2

A microbial preparation was prepared by the same process as shown in Example 1 with an exception of using Bacillus siamensis KS-R02 (KCTC 13559BP) as a microbial starter.

### Example 3

A microbial preparation was prepared by the same process as shown in Example 1 with an exception of using Bacillus velezensis KS-R03 (KCTC 13560BP) as a microbial starter.

### Example 4

A microbial preparation was prepared by the same process as shown in Example 1 with an exception of using Bacillus tequilensis KS-R04 (KCTC 13561BP) as a microbial starter.

### Example 5

A microbial preparation was prepared by the same process as shown. in Example 1 with an exception of using a mixed strain obtained by mixing Bacillus amyloliquefaciens KS-R01 (KCTC 13558BP), Bacillus siamensis KS-R02 (KCTC 13559BP), Bacillus velezensis KS-R03 (KCTC 13560BP) and Bacillus tequllensis KS-R04 (KCTC 13561BP) as a microbial starter in the same amount.

### Experimental Example 2

### Experimental Method

As for the conditions for injecting the microbial preparation, an activating solution, which was the 20-fold dilution of the stock solution of the complex microbial preparation, was injected 1/50 compared to the volume of the soil, so as to exert an optimal performance.

A soil sample was collected from the soil of the Danyang area, which a granite base with the amount of radon generated exceeding a reference value, and an average value of naturally occurring radon in the soil collected from the area is about 180 becquerels (Bq/m³) as measured by a radon meter.

### Experimental Results

TABLE 8
Radon value measurement results (First)

Radon value (Bq/m³)

| Classification | 0 | 60 min | 120 min | 180 min |
|---|---|---|---|---|
| Example 1 | 182 | 166 | 125 | 86 |
| Example 2 | 179 | 165 | 122 | 82 |
| Example 3 | 183 | 164 | 123 | 80 |
| Example 4 | 184 | 166 | 120 | 79 |
| Example 5 | 183 | 142 | 108 | 58 |
| Comparative Example | 182 | 179 | 172 | 168 |

Table 8 shows the results of continuously investigating a radon value generated from the soil sample at an interval of 60 minutes during an experiment using a radon meter. From the above experimental results, it can be confirmed that the microbial preparation according to the present invention significantly decreases the amount of radon generated, and thus it is expected to be very effective in preventing the environmental pollution of radioactively contaminated areas and restoring the environment.

As described above, the present invention has been described with reference to preferred exemplary embodiments herein, but it will be understood by those skilled in the art that the present invention may be variously changed and modified without departing from the spirit and field of the present invention, as described in the following scope of patent claims.

SEQ ID NO: 1: 16S sequence of T16-11k (Bacillus
amyloliquefaciens)
[96]GTGACAGGTGGTGCATGGTTGTCGTCAGCTCGTGTCGTGAGATGT
TGGGgTTAAGTCCCGCAACGAGCGCAACCCTTGATCTTAGTTGCCAGCA
TTCAGTTGGGCACTCTAAGGTGACTGCCGGTGACAAACCGGAGGAAGGT
GGGGATGACGTCAAATCATCATGCCCCTTATGACCTGGGCTACACACGT
GCTACAATGGGCAGAACAAAGGGCAGCGAAACCGCGAGGTTAAGCCAAT
CCCACAAATCTGTTCTCAGTTCGGATCGCAGTCTGCAACTCGACTGCGT
GAAGCTGGAATCGCTAGTAATCGCGGATCAGCATGCCGCGGTGAATACG
TTCCCGGGCCTTGTACACACCGCCCGTCACACCACGAGAGTTTGTAACA
CCCGAAGTCGGTGAGGTAACCTTTTTGGAGCCAGCCGCCGAAGGTGGGA
CAGATGATTGGGGTG
SEQ ID NO: 2: 16S sequence of T12-11k (Bacillus
siamensis)
GTGACAGGTGGTGCATGGTTGTCGTCAGCTCGTGTCGTGAGATGTTGGG
TTAAGTCCCGCAACGAGCGCAACCCTTGATCTTAGTTGCCAGCATTCAG
TTGGGCACTCTAAGGTGACTGCCGGTGACAAACCGGAGGAAGGTGGGGA
TGACGTCAAATCATCATGCCCCTTATGACCTGGGCTACACACGTGCTAC
AATGGACAGAACAAAGGGCAGCGAAACCGCGAGGTTAAGCCAATCCCAC
AAATCTGTTCTCAGTTCGGATCGCAGTCTGCAACTCGACTGCGTGAAGC
tGGAATCGCTAGTAATCGCGGATCAGCATGCCGCGGTGAATACGTTCCC
GGGCCTTGTACACACCGCCCGTCACACCACGAGAGTTTGTAACACCCGA
AGTCGGTGAGGTAACCTTTATGGAGCCAGCCGCCGAAGGTGGGACAGAT
GATTGGGGTG
SEQ ID NO: 3: 16S sequence of T19-8k (Bacillus
velezensis)
GTGACAGGTGGTGCATGGTTGTCGTCAGCTCGTGTCGTGAGATGTTGGG
TTAAGTCCCGCAACGAGCGCAACCCTTGATCTTAGTTGCCAGCATTCAG
TTGGGCACTCTAAGGTGACTGCCGGTGACAAACCGGAGGAAGGTGGGGA
TGACGTCAAATCATCATGCCCCTTATGACCTGGGCTACACACGTGCTAC
AATGGACAGAACAAAGGGCAGCGAAACCGCGAGGTTAAGCCAATCCCAC
AAATCTGTTCTCAGTTCGGATCGCAGTCTGCAACTCGACTGCGTGAAGC
TGGAATCGCTAGTAATCGCGGATCAGCATGCCGCGGTGAATACGTTCCC
GGGCCTTGTACACACCGCCCGTCACACCACGAGAGTTTGTAACACCCGA
AGTCGGTGAGGTAACCTTTTAGGAGCCAGCCGCCGAAGGTGGGACAGAT
GATTGGGGTG
SEQ ID NO: 4: 16S sequence of Y11-6k (Bacillus
tequilensis)
[105]GTGACAGGTGGTGCATGGTTGTCGTCAGCTCGTGTCGTGAGATG
TTGGGTTAAGTCCCGCAACGAGCGCAACCCTTGATCTTAGTTGCCAGCA
TTCAGTTGGGCACTCTAAGGTGACTGCCGGTGACAAACCGGAGGAAGGT
GGGGATGACGTCAAATCATCATGCCCCTTATGACCTGGGCTACACACGT
GCTACAATGGACAGAACAAAGGGCAGCGAAACCGCGAGGTTAAGCCAAT
CCCACAAATCTGTTCTCAGTTCGGATCGCAGTCTGCAACTCGACTGCGT
GAAGCTGGAATCGCTAGTAATCGCGGATCAGCATGCCGCGGTGAATACG
TTCCCGGGCCTTGTACACACCGCCCGTCACACCACGAGAGTTTGTAACA
CCCGAAGTCGGTGAGGTAACCTTTTAGGAGCCAGCCGCCGAAGGTGGGA
CAGATGATTGGGGTG

1. A biological microbial treating agent for radioactive material removal, comprising at least one type of microorganismselected from Bacillus amyloliquefaciens KS-R01, Bacillus siamensis KS-R02, Bacillus lezensis KS-R03 and Bacillus tequilensis KS-R04.

2. The biological microbial treating agent of claim comprising Bacillus amyloliquefaciens KS-R01, Bacillus siamensis KS-R02, Bacillus velezensis KS-R03 and Bacillus tequilensis KS-R04.

3. A method for removing radioactive materials by treating water or soil contaminated with radioactive materials with the microbial treating agent of claim 1.
